(19) United States
(12) Patent Application Publication
(10) Pub. No.: US 2002/0164267 A1
(43) Pub. Date: Nov. 7, 2002

(54) Method of preventing type 2 diabetes with aerosolized insulin

(76) Inventor: Alan S. Krasner, East Lyme, CT (US)

Correspondence Address: PFIZER INC., PATENT DEPARTMENT, MS8260-1611, EASTERN POINT ROAD, GROTON, CT, 06340, US

(21) Appl. No.: 10/137,469

(22) Filed: Apr. 30, 2002

Related U.S. Application Data
(60) Provisional application No. 60/288,732, filed on May 4, 2001.

(51) Int. Cl. A61M 1/14; A61M 1/34; A61M 1/36; A61M 11/00

(52) U.S. Cl. 422/46

(57) ABSTRACT

Inhaled insulin can be used to prevent the progression of beta cell failure in Type 2 diabetes, thereby slowing the onset, or by stopping the onset indefinitely, or ameliorating the disease in patients in whom it is already established.

### FIELD OF THE INVENTION

This invention is directed to a method of preventing Type 2 diabetes, with inhaled insulin, comprising administering, to a human patient in need of such prevention, an effective amount of inhaled insulin. The method operates, inter alia, by slowing or arresting the immunologically mediated progression of pancreatic beta cell failure.

### BACKGROUND OF THE INVENTION

Diabetes mellitus is a serious metabolic disease that is defined by the presence of chronically elevated levels of blood glucose. Classic symptoms of diabetes mellitus in adults are polyuria and polydipsia together with elevated levels of plasma glucose. Normal fasting plasma glucose concentrations are less than 110 milligrams per deciliter. In diabetic patients, fasting concentrations are found to be at or above 126 milligrams per deciliter. In general, diabetes mellitus develops in response to damage to, or to defects in, the beta cells of the pancreas.

Primary diabetes mellitus is classified as Type 1 diabetes (also called insulin-dependent diabetes mellitus or IDDM) and Type 2 diabetes mellitus (also called non-insulin dependent diabetes mellitus or NIDDM). Type I (juvenile onset or insulin-dependent) diabetes is a well-known hormone deficient state, in which the pancreatic beta cells appear to have been destroyed by the body's own immune defense mechanisms. Patients with Type I diabetes mellitus have little or no endogenous insulin secretory capacity. These patients develop extreme hyperglycemia. Type I diabetes was fatal until the introduction of insulin replacement therapy some 70 years ago—first using insulins from animal sources, and more recently, using human insulin made by recombinant DNA technology. The destruction of beta cells in Type I diabetes leads to the inability to produce insulin, and thereby a chronic insulin deficiency. To these types of patients, insulin is most commonly administered by subcutaneous injection, typically into the abdomen or upper thighs.

Type 2 diabetes is characterized by insulin resistance, i.e., a failure of the normal metabolic response of peripheral tissues to the action of insulin. In other words, insulin resistance is a condition where the circulating insulin produces a subnormal biological response. In clinical terms, insulin resistance is present when normal or elevated blood glucose levels persist in the face of normal or elevated levels of insulin. The hyperglycemia associated with Type 2 diabetes can sometimes be reversed or ameliorated by diet or weight loss sufficient to restore the sensitivity of the peripheral tissues to insulin. Progression of Type 2 diabetes mellitus is associated with increasing concentrations of blood glucose and coupled with a relative decrease in the rate of glucose-induced insulin secretion. Thus, for example, in late-stage Type 2 diabetes mellitus, there may be an insulin deficiency. There is evidence for an autoimmune pathology in a subset of type 2 diabetics, namely the subclass which has come to be known as “latent autoimmune diabetes in adults”, abbreviated as LADA.

The nature of the lesion of the pancreatic beta cells in Type 2 diabetes is not clear. Unlike the pancreatic beta cells in Type I diabetics, the beta cells of Type 2 diabetics retain the ability to synthesize and secrete insulin and amylin.

The timewise progressions of Type I versus Type 2 diabetes can (and usually do) differ markedly. Youthful (e.g., pediatric) patients suffering from Type I diabetes may not have their condition diagnosed until after the bulk of pancreatic beta cells have been destroyed, thereby necessitating chronic insulin therapy. Usually, Type I progresses on the order of a couple of years before the pancreas has been damaged to the point that it no longer produces sufficient insulin to meet the patient's metabolic needs.

The treatment of Type I diabetes thus necessarily involves the administration of replacement doses of insulin, administered by the parenteral route. In combination with the correct diet and self-blood glucose monitoring, the majority of Type I diabetics can achieve a certain level of control of blood glucose.

In contrast to Type I diabetes, treatment of Type 2 diabetes frequently does not require the use of insulin, and the condition itself can progress over several decades. Institution of therapy in Type 2 diabetes usually involves a trial of dietary therapy and lifestyle modification, typically for 6-12 weeks in the first instance. Features of a diabetic diet include an adequate but not excessive total calorie intake, with regular meals, restriction of the content of saturated fat, a concomitant increase in the polyunsaturated fatty acid content, and an increased intake of dietary fiber. Lifestyle modifications include the maintenance of regular exercise, as an aid both to weight control and also to reduce the degree of insulin resistance. If after an adequate trial of diet and lifestyle modifications, fasting hyperglycemia persists, then a diagnosis of “primary diet failure” may be made, and either a trial of oral hypoglycemic therapy or direct institution of insulin therapy will be required to produce blood glucose control and, thereby, to minimize the complications of the disease. Type 2 diabetics who fail to respond to diet and weight loss may respond to therapy with oral non-insulin hypoglycemic agents such as sulfonylureas or biguanides. Insulin therapy, however, is used to treat other patients with Type 2 diabetes, especially those who have undergone primary dietary failure and are not obese, or those who have undergone both primary diet failure and secondary oral hypoglycemic failure.

### SUMMARY OF THE INVENTION

This invention provides a method of preventing Type 2 diabetes with inhaled insulin, comprising administering to a human patient in need of such prevention, an effective amount of inhaled aerosolized insulin. Thus the insulin itself is an aerosolized, particulate insulin, and it is inhaled, that is, it is administered by the pulmonary (deep lung) route.

“Insulin” means the art-recognized polypeptide used in the treatment of diabetics in a substantially purified form, and also the various commercially available forms which include excipients. The term encompasses natural extracted human insulin, recombinantly produced human insulin, insulin extracted from bovine and/or porcine sources, recombinantly produced porcine and bovine insulin, and mixtures thereof. The term “insulin” is also intended to encompass insulin analogs wherein one or more of the amino acids within the polypeptide chain has been replaced with an alternative amino acid and/or wherein one or more of the amino acids has been deleted or wherein one or more additional amino acids has been added to the polypeptide chain. In general, such insulin analogs of the present invention include “super insulin analogs” wherein the ability of the insulin analog to affect serum glucose levels is substantially enhanced as compared with conventional insulin as well as hepatoselective insulin analogs which are more active in the liver than in adipose tissue. The invention may employ an aerosolized inhaled insulin which is monomeric, such as insulin lispro.

“Inhaled insulin” means an aerosolized, wet or dry, particulate, insulin-containing formulation which is administered via the pulmonary route by having the patient “breathe” an insulin-containing aerosol into the lungs, generally by drawing the aerosol through the mouth and into the lungs. The formulation can, for example, comprise dry particles inhaled from a dry powdered inhaler such as that available from Inhale Therapeutics Systems, San Carlos, Calif. The inhaled insulin formulation can also be a particulate, insulin-containing formulation suspended in a propellant. Alternatively, the formulation can be a wet aerosol, i.e., a liquid aerosol of the type produced from an aqueous solution of insulin by a liquid nebulizer system (see Laube, Journal of Aerosol Medicine, Vol 4, No. 3, 1991, and U.S. Pat. No. 5,320,094, herein incorporated by reference in their entirety). The exact aerosol formulation is not believed to be particularly critical, such that the powder can be in the form of a dry powder or a wet insulin-containing aerosol of the type produced by a nebulizer, so long as the particle size, whether liquid or dry, is of a size which facilitates penetration to the deep lung, in which it is believed that the alveoli serve as the portals from the lungs to the blood. Generally, such a particle size is less than about 10 μm. “Inhaled insulin” is to be contrasted with “intranasally administered insulin” in which insulin is administered within the nasal passages and is weakly absorbed into the bloodstream through the nasal mucosa.

Inhaled insulin, in this invention, is employed to “prevent” diabetes, “prevention” meaning that an insulin-containing aerosol can be administered to patients prior to the onset of clinically apparent Type 2 diabetes in order to delay that onset by a period ranging anywhere from a few months or years, up to essentially permanently. In this mode of prevention, a physician will generally diagnose that a patient is at risk for developing Type 2 diabetes by diagnosing that the patient has any one or more of the following conditions which are considered “precursor states” signalling the future onset of actual Type 2 diabetes.

1. Impaired fasting glucose, meaning that the patient does not exhibit a normal fasting glucose concentration of less than 110 mg/dl nor does the patient exhibit a fasting concentration of 126 mg/dl or above. In this situation the patient is considered to have impaired fasting glucose, but is not outright diagnosable as diabetic.

2. Impaired glucose tolerance, i.e. a state in which the patient does not respond normally when administered a standard oral glucose tolerance test.

Impaired glucose tolerance is defined as a glucose level between 140-199 mg/dl two hours after the oral intake of 75 grams of glucose.

3. Women with a history of gestational diabetes but who are not yet diabetic. In this precursor state the female patient would qualify or be diagnosed as diabetic at one or more times during pregnancy, but would not be diagnosed as diabetic outside of pregnancy.

“Prevention” in this invention also means that a patient can be administered inhaled insulin to slow or even completely arrest the progression of Type 2 diabetes once it has become clinically apparent and/or after it has been diagnosed. That is, a patient may be diagnosed as having Type 2 diabetes in an early stage such that the administration of exogenous insulin to control sugar metabolism is not yet required. Inhaled insulin may nonetheless be prescribed at this point, not to control glucose metabolism, but to slow and/or arrest the progression of the Type 2 diabetes through its immunological effects. In this regard, it is noted that Type 2 diabetes may in many cases be characterized by the gradual death of pancreatic beta cells over time through an immunological mechanism, i.e, by lymphocytes which slowly, over many years and even decades, effect or aid in the destruction of pancreatic beta cells and thereby bring about the onset and progression of Type 2 diabetes. Administration of inhaled insulin is believed to act, inter alia, by suppressing the activity of such lymphocytes. Thus, some patients who take inhaled insulin may have the progression of their Type 2 diabetes slowed, with the advantage that such patients can delay having to take insulin simply to metabolize glucose. Instead, such patients will be able to rely on diet, exercise, and/or non-insulin therapy for much longer than in the absence of inhaled insulin therapy. Other patients may have the progression of their Type 2 diabetes arrested, with the result that they may never need to take exogenous insulin to control their blood sugars.

It is preferred to administer inhaled insulin from the time that a patient is either diagnosed to be at risk (i.e. to exhibit one or more of the aforementioned precursor states) or diagnosed to actually have Type 2 diabetes, at least up to the time that exogenous insulin administration becomes required to aid in controlling sugar metabolism.

To obtain the benefits of inhaled insulin through its immunological effects of preserving pancreatic beta cells, it is preferred to commence the administration of inhaled insulin to a patient as far in advance as possible of having to chronically administer insulin to control glucose metabolism. This means that inhaled insulin would be administered starting when either a precursor state or the actual onset of Type 2 diabetes is diagnosed by the physician. Even if Type 2 diabetes is not detected until later in its progression, say until after it has become necessary to administer insulin for glucose control, inhaled insulin can still be administered, to preserve or to at least slow the destruction of whatever beta cell function remains, in addition to controlling blood sugar metabolism.

Thus, the invention provides the means to prevent Type 2 diabetes in patients who exhibit a precursor state indicating that they are at risk for developing this condition. As mentioned above, the immunological capability of inhaled insulin to prevent Type 2 diabetes is thought to be associated with inhaled insulin's ability to preserve, or at least retard, the damage to pancreatic beta cells, which damage would otherwise lead to the onset and/or progression of Type 2 diabetes. The capability for prevention also derives from inhaled insulin's ability to arrest or slow pancreatic beta cell damage once Type 2 diabetes onset has occured.

To summarize, the “prevention” afforded by the present invention may manifest itself in one or more of the following ways:

1. a slowing of the onset of Type 2 diabetes when it is detected prior to clinical onset. This will occur when a regimen of inhaled insulin is initiated immediately after detection of a precursor state, or at least before the actual clinical onset of Type 2 diabetes. In patients who have already had the onset of clinical diabetes, in most if not all such patients, the condition will also be more stable in the sense that blood glucose levels fluctuate less and exhibit less variability relative to the fluctuations and variability occurring in the absence of inhaled insulin therapy. In addition, less non-inhaled insulin therapy (exercise, diet control and other non-inhaled insulin antidiabetic medications) would be required.

2. in some cases when a precursor state is detected prior to clinical onset, the progression to Type 2 diabetes can be prevented altogether, meaning that the patient will never require any form of diabetic therapy, including exercise, dietary control oral agents, or exogenous insulin administration, beyond that which is inhaled, to effect prevention.

3. a slowing, and in some patients the complete stoppage, of the progression of diabetes by inhaled insulin in patients in whom the condition is clinically apparent.

The invention is surprising in that it represents a total change to the current practice of treating clinical Type 2 diabetes through diet, exercise, and/or medications which control the blood sugar supply through other than immunological means. As mentioned above, examples of non-insulin medications which can be used to control blood sugar levels are the sulfonylureas and biguanides, known to the art, which are employed to restore normal glucose via a variety of non-immunological mechanisms.

### DETAILED DESCRIPTION

A. Inhalers/Administration

Any inhaler known to the art may be used for this invention so long as it is capable of delivering a therapeutically effective dose of insulin to the deep lung. This includes any device such as those which are classified as dry powder inhalers, nebulizers, and metered dose inhalers. Potentially useful are art-recognized inhalers such as those sold under the names Turbohaler (Astra), Rotahaler® (Glaxo), Diskus® (Glaxo), the Ultravent nebulizer (Mallinckrodt), the Acorn II nebulizer (Marquest medical Products), the Ventolin® metered dose inhaler (Glaxo), the Spinhaler® powder inhaler (Fisons), or the like.

In a preferred embodiment, insulin is inhaled as a dry powder by means of a hand-held device such as that disclosed in any of U.S. Pat. Nos. 6,089,228, 5,458,135, 5,775,320, 5,785,049, 5,740,794, and WO 93/00951, the full disclosures of which are herein incorporated by reference. Such devices are available from Inhale Therapeutics Systems, San Carlos, Calif.

B. Formulations

Any formulation which makes it possible to produce aerosolized forms of insulin which can be inhaled and delivered to a patient via the intrapulmonary route can be used in connection with the present invention. Specific information regarding formulations which can be used in connection with aerosolized delivery devices are described within Remington's Pharmaceutical Sciences, A. R. Gennaro editor (latest edition) Mack Publishing Company. Regarding insulin formulations, it is also useful to note Sciarra et al. [Journal of Pharmaceutical Sciences, Vol. 65, No. 4, 1976].

A variety of different insulin-containing aerosol formulations can be used in connection with the present invention. The active ingredient within such formulations is insulin which is preferably recombinantly produced human insulin, but which may include insulin extracted from animal sources. Further, the insulin may be an insulin analog which is an analog of human insulin which has been recombinantly produced. Although the insulin and/or analog may be present by itself as the sole active ingredient, the insulin may be present with an additional active ingredient such as a sulfonylurea. However, such sulfonylureas are generally administered separately in order to more closely control dosing and serum glucose levels.

Regardless of the active ingredient, there are several basic types of insulin formulations which can be used in connection with the present invention. All of the formulations include insulin, preferably with a pharmaceutically acceptable carrier suitable for intrapulmonary administration. In accordance with a first formulation, a low boiling point, highly volatile propellant is combined with the active ingredient and a pharmaceutically acceptable excipient. The active ingredient may, for example, be provided as a suspension or dry powder in the propellant, or the active ingredient may be dissolved in solution within the propellant. Both of these formulations may be readily included within a container which has a valve as its only opening. Since the propellant is highly volatile, i.e., has a low boiling point, the contents of the container will be under pressure. Thus, when low boiling point propellants are used, the propellants are held within a pressurized canister of the device and maintained in a liquid state. When the valve is actuated, the propellant is released and forces the active ingredient from the canister along with the propellant. The propellant will “flash” upon exposure to the surrounding atmosphere, i.e., the propellant immediately evaporates. The flashing occurs so rapidly that it is essentially pure active ingredient which is actually delivered to the lungs of the patient. The “flashing” phenomenon which occurs with the use of low boiling point propellents may greatly increase the practicality of the present invention as compared with nebulizers or formulations which do not use such propellants in that larger amounts of drug can be easily administered in a short period of time. Further, since the material being delivered to the lungs is essentially pure drug, it is easier to monitor and more closely control dosing which is a critical feature of the methodology of the present invention. Accordingly, when using such a delivery device it is preferable to use low boiling point propellants such as low boiling point chlorofluorocarbons or hydrocarbons, e.g., trichlorofluoromethane and dichlorodifluoromethane. As non-chlorofluorocarbon containing propellants are developed which are low boiling point propellants, their use in connection with the present invention will become apparent to those skilled in the art.

In accordance with a second formulation, the insulin is provided in a solution formulation. In this embodiment a dry powder is preferably dissolved in an aqueous solvent to create a solution which is moved through a porous membrane to create an aerosol for inhalation. Such solutions can be of the type which are made available commercially for injection and/or other solutions which are more acceptable for intrapulmonary delivery. An example of a suitable solution for generating aqueous aerosols from a nebulizer is the 0.9% saline solution disclosed in Laube, U.S. Pat. No. 5,320,094.

A preferred form for inhaled administration of insulin is as a dry powder. Preferred insulin dry powders include those which are described in U.S. Pat. No. 5,997,848 to Patton et al. Such insulin powders comprise free flowing particulates having a size selected to permit penetration into the alveoli of the lungs, generally being less than 10 μm in diameter, preferably less than 7.5 μm, most preferably less than 5 μm, and usually being in the range from 0.1 μm to 5 μm in diameter. The aforementioned particle sizes generally apply to solid particles. It is also feasible to employ larger size particles which are aerodynamically light but which have a mean diameter much larger than 10 um, say 5 to 30 um. Such particles generally have a low tap density, less than 0.4 g/cm, and a mean diameter between 1 and three microns. Such particles are disclosed in US patent RE37,053 E, and in PCT published application WO 01/13891, both herein incorporated by reference. In any case, the insulin powder employed should be of a size that is adapted to penetrating to the deep lung where it can be absorbed through the alveoli.

Alternatively, amorphous insulins could be prepared by lyophilization (freeze-drying), vacuum drying, or evaporative drying of a suitable insulin solution under conditions to produce the amorphous structure. The amorphous insulin so produced can then be ground or milled to produce particles within the desired size range. Crystalline dry powder insulins may be formed by grinding or jet milling of bulk crystalline insulin. The preferred method for forming insulin powders comprising particulates in the desired size range is spray drying, where pure, bulk insulin (usually in a crystalline form) is first dissolved in a physiologically acceptable aqueous buffer, typically a citrate buffer having a pH in the range from about 2 to 9. The insulin is dissolved at a concentration from 0.01% by weight to 1% by weight, usually from 0.1% to 0.2%. The solutions may then be spray dried in conventional spray drying equipment from commercial suppliers, such as Buchi, Niro, and the like, resulting in a substantially amorphous particulate product.

The dry insulin powders may consist essentially of insulin particles within the requisite size range and be substantially free from any other biologically active components, pharmaceutical carriers, and the like. Such “neat” formulations may include minor components, such as preservatives, present in low amounts, typically below 10% by weight and usually below 5% by weight. Using such neat formulations, the number of inhalations required for even high dosages can be substantially reduced, often to only a single breath.

The insulin powders useful in the present invention may optionally be combined with pharmaceutical carriers or excipients which are suitable for respiratory and pulmonary administration. Such carriers may serve simply as bulking agents when it is desired to reduce the insulin concentration in the powder which is being delivered to a patient, but may also serve to enhance the stability of the insulin compositions and to improve the dispersability of the powder within a powder dispersion device in order to provide more efficient and reproducible delivery of the insulin and to improve handling characteristics of the insulin such as flowability and consistency to facilitate manufacturing and powder filling.

Suitable carrier materials may be in the form of an amorphous powder, a crystalline powder, or a combination of amorphous and crystalline powders. Suitable materials include carbohydrates, e.g. monosaccharides such as fructose, galactose, glucose, D-mannose, sorbose, and the like; disaccharides, such as lactose, trehalose, cellobiose, and the like; cyclodextrins, such as 2-hydroxypropyl-β-cyclodextrin; and polysaccharides, such as raffinose, maltodextrins, dextrans, and the like; (b) amino acids, such as glycine, arginine, aspartic acid, glutamic acid, cysteine, lysine, and the like; (c) organic salts prepared from organic acids and bases, such as sodium citrate, sodium ascorbate, magnesium gluconate, sodium gluconate, tromethamine hydrochloride, and the like; (d) peptides and proteins, such as aspartame, human serum albumin, gelatin, and the like; (e) alditols, such as mannitol, xylitol, and the like. A preferred group of carriers includes lactose, trehalose, raffinose, maltodextrins, glycine, sodium citrate, tromethamine hydrochloride, human serum albumin, and mannitol.

Such carrier materials may be combined with the insulin prior to spray drying, i.e., by adding the carrier material to the buffer solution which is prepared for spray drying. In that way, the carrier material will be formed simultaneously with and as part of the insulin particles. Typically, when the carrier is formed by spray drying together with the insulin, the insulin will be present in each individual particle at a weight percent in the range from 5% to 95%, preferably from 20% to 80%. The remainder of the particle will primarily be carrier material (typically being from 5% to 95%, usually being from 20% to 80% by weight), but will also include buffer(s) and may include other components as described above. The presence of carrier material in the particles which are delivered to the alveolar region of the lung (i.e., those in the requisite size range below 10 μm) has been found not to significantly interfere with systemic absorption of insulin.

Alternatively, the carriers may be separately prepared in a dry powder form and combined with the dry powder insulin by blending. The separately prepared powder carriers will usually be crystalline (to avoid water absorption), but might in some cases be amorphous or mixtures of crystalline and amorphous forms. The size of the carrier particles may be selected to improve the flowability of the insulin powder, typically being in the range from 25 μm to 100 μm. Carrier particles in this size range will generally not penetrate into the alveolar region of the lung and will often separate from the insulin in the delivery device prior to inhalation. Thus, the particles which penetrate into the alveolar region of the lung will consist essentially of insulin and buffer. A preferred carrier material is crystalline mannitol having a size in the above-stated range.

The dry insulin powders useful in the present inventions may also be combined with other active components. For example, it may be desirable to combine small amounts of amylin or active amylin analogues in the insulin powders to improve the treatment of diabetes. Amylin-is a hormone which is secreted with insulin from the pancreatic β-cells in normal (non-diabetic) individuals. It is believed that amylin modulates insulin activity in vivo, and it has been proposed that simultaneous administration of amylin with insulin could improve blood glucose control. Combining dry powder amylin with insulin in the compositions of the present invention will provide a particularly convenient product for achieving such simultaneous administration. Amylin may be combined with insulin at from 0.1% by weight to 10% by weight (based on the total weight of insulin in a dose), preferably from 0.5% by weight to 2.5% by weight. Amylin is available from commercial suppliers, such as Amylin Corporation, San Diego, Calif., and can be readily formulated in the compositions of the present invention. For example, amylin may be dissolved in aqueous or other suitable solutions together with the insulin, and optionally carriers, and the solution spray dried to produce the powder product.

The dry powder insulin compositions of the present invention are preferably aerosolized by dispersion in a flowing air or other physiologically acceptable gas stream in a conventional manner. One system suitable for such dispersion is described in copending application Ser. No. 07/910,048, which-has been published as WO 93/00951, the full disclosures of which are incorporated herein by reference. The full operation of such a device is disclosed therein.

A preferred dry powder formulation, particularly for use with the aforementioned inhaler, is disclosed in WO 98/16205 (the full text of which is herein incorporated by reference) as example 3. It consists of a dry powder made by spray drying a formulation containing 7.50 mg insulin, 1.27 mg mannitol, 3.38 mg sodium citrate, 0.026 mg sodium hydroxide, and 0.32 mg glycine per milliliter of deionized water, for a total solids concentration of 12.5 mg/mL at pH 7.3, the formulation being spray dried to produce a dry powder having an average particle size less than 5 μm. The powder is delivered to the deep lung via an inhaler. The preferred mode of treatment with dry powder insulin is as described in the aforementioned U.S. Pat. No. 5,997,848.

C. Tests

The tests used herein to assess the state of a patient, and whether or not a regimen of inhaled insulin is appropriate, are the well known, art-recognized measurement of fasting and/or random serum, plasma, or whole blood glucose measurements. Assessment of beta cell function can be accomplished using glucose tolerance tests, especially intravenous glucose tolerance testing. In this method, glucose concentrations and plasma or serum insulin concentrations (the latter usually measured by radioimmunoassay or related techniques) are sampled before and after the administration of a known amount of glucose orally or intravenously. There are known normal patterns of glucose/insulin response to glucose challenge. Subjects with beta-cell dysfunction due to diabetes precursor states or diabetes will generally exhibit abnormal glucose/insulin responses to glucose challenge. Laboratory methods for measuring glucose and insulin levels are widely commercially available.

As a less preferred alternative, C-peptide concentrations (measured by radioimmunoassay or related techniques) in the fasting state and/or after glucose challenge (oral or intravenous) can be used to assess the functional integrity of pancreatic beta cells.

The amount of insulin to be administered and the appropriate regimen will generally be determined by the attending physician. In general, when administering insulin in the form of an aerosol, as recombinantly produced human insulin plus excipients, a patient will be administered an amount of inhaled insulin in the range of 0.5 to 50 mg per day, usually 0.5 to 25 mg per day, usually in 1 to 4 individual dry powder doses. The dosing event itself will usually include administering the required dose in one or more, usually 1 to 4, inhalations from a suitable inhaler or nebulizer. Regardless of the form in which the inhaled insulin is delivered, i.e., regardless of whether the insulin is delivered as a dry powder, as an aqueous aerosol produced by a nebulizer, or as a suspension in a propellant delivered from a metered dose inhaler, a patient will be delivered an amount of insulin equivalent to between 1.5 and 150 units delivered systemically (1 mg of inhaled insulin being generally equivalent to about 3 Units (U) of systemically delivered recombinant human insulin). Insulin analogs which are superactive can be administered in substantially smaller amounts while obtaining substantially the same effect with respect to reducing serum glucose levels.

Diabetes prevention induced by inhaled insulin can be demonstrated in animals predisposed to the development of spontaneous diabetes. A suitable animal test model is disclosed in Harrison et al (J. Exp. Med., 184: 2167-2174, 1996) in which semisealed boxes containing groups of eight Nonobese Diabetic (NOD) mice were exposed to aerosolized recombinant human insulin by connection to a standard patient electric pump and a nebulizer. Other strains of mice and other species may also be appropriate for this purpose. In such animals, after inhaled insulin exposure, time to onset of hyperglycemia in inhaled insulin treated animals can be compared to that in control air-exposed animals. After a (usually pre-determined) period of exposure, pancreatic histology can be evaluated for cellular inflammatory autoimmune activity. In vitro, reduced lymphocyte proliferative responses to autoantigens can be shown to correlate with reduced pancreatic lymphocytic inflammation. Examples of relevant autoantigens which can be used in these lymphocyte proliferation assays include: insulin, insulin denaturation products or major epitopes, and related islet antigens. Cytokines (such as interleuken-4 and interleuken-10) levels can be measured in inhaled insulin vs control animals, and can be used to assess degrees of anti-inflammatory cellular immune responses.

What is claimed is:

1. A method of preventing immunologically mediated beta cell failure prior to or after the onset of Type 2 diabetes with inhaled insulin, comprising administering to a human patient in need of such prevention, an effective amount of inhaled insulin.

2. A method as defined in claim 1, wherein said inhaled insulin is delivered in the form of aerosolized insulin wherein the aerosol is atomized from a solution.

3. A method as defined in claim 1, wherein said inhaled insulin is administered as a dry powder.

4. A method as defined in claim 1, wherein said effective amount of inhaled insulin is delivered by a propellant as a solution or suspension of said insulin from a metered dose inhaler.

5. A method as defined in claim 1, wherein said inhaled insulin is administered from the time that a patient is diagnosed to be either at risk or to actually have Type 2 diabetes up to the time that exogenous insulin administration becomes required to aid in controlling sugar metabolism.
